SEQUENCE LISTING (4 sequences)

SEQ ID NO: 1 - DNA, 1719 bp, Artificial Sequence
  Synthesized Sequence
atgccgcgcaaaatgtatagctgcgactttgaaaccaccaccaaagtggaagattgccgcgtttgggcgtatggctatatgaacatcgaagaccacagcgaatacaaaattggcaacagcctggatgaatttatggcgtgggtgctgaaagttcaggcggatctgtattttcacaacctgaaatttgacggcgcgttcattattaactggctggaacgcaacggctttaaatggagcgcggatggcttaccgaacacctataacaccattattagccgcatgggccagtggtatatgattgatatctgcctgggctataaaggcaaacgcaagattcataccgtgatctatgatagcctgaagaaactgccgtttccggtgaaaaaaatcgcgaaggactttaaactgaccgtgctgaaaggcgatattgactaccataaagaacgcccggtgggctataaaattaccccggaggaatatgcgtacatcaagaacgacatccagattattgcggaagcgctgctgattcagtttaaacagggcctggatcgtatgaccgcgggtagcgatagcctgaaaggctttaaggacattatcaccaccaagaagttcaagaaagtgtttccgaccctgagcctgggcctggataaagaagtgcgctatgcgtatcgcggtggctttacctggctgaacgatcgctttaaggaaaaggaaattggcgaaggcatggtgtttgatgtgaacagcctgtatccggcgcagatgtatagccgcctgctgccgtatggtgaaccgattgtgtttgaaggcaagtatgtgtgggatgaagattatccgctgcacattcagcatattcgctgcgaattcgaactgaaggaaggctatattccgaccattcagattaaacgcagccgcttttataaaggcaacgagtacctgaaaagcagcggcggcgaaattgcggatctgtggctgagcaacgtggatctggaactgatgaaagaacactacgatctgtacaacgtggaatatatcagcggcctgaaatttaaagcgaccaccggcctgtttaaggactttatcgacaagtggacctacattaaaaccaccagcgaaggcgcgattaaacagctggcgaaactgatgctgaacagcctgtatggcaaatttgcgagcaacccggatgttaccggcaaagtgccgtatctgaaagaaaacggcgcgctgggctttcgtttaggcgaagaggaaaccaaagatccggtgtataccccgatgggcgtgtttattaccgcgtgggcgcgctataccaccattaccgcggcgcaggcgtgttatgatcgcattatctattgcgataccgatagcattcatctgaccggcaccgaaattccggatgtgatcaaagatattgtggacccgaaaaaactgggctattgggcgcatgaaagcacctttaaacgcgcgaaatatctgcgccagaaaacctatatccaggacatctacatgaaagaggtggatggcaaactggttgaaggcagcccggatgattataccgatattaagttcagcgtgaaatgcgcgggcatgaccgataaaattaagaaggaagtgaccttcgagaactttaaagtgggctttagccgcaaaatgaaaccgaaaccggttcaggtgcctggcggtgttgttctggtggatgataccttcaccatcaagtga

SEQ ID NO: 2 - protein, 572 aa, Artificial sequence
  Synthesized Sequence
MPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 3 - DNA, 1779 bp, Artificial sequence
  Synthesized Sequence
atgggcagcagccatcatcatcatcatcacagcagcggcctggtgccgcgcggcagccatatgccgcgcaaaatgtatagctgcgactttgaaaccaccaccaaagtggaagattgccgcgtttgggcgtatggctatatgaacatcgaagaccacagcgaatacaaaattggcaacagcctggatgaatttatggcgtgggtgctgaaagttcaggcggatctgtattttcacaacctgaaatttgacggcgcgttcattattaactggctggaacgcaacggctttaaatggagcgcggatggcttaccgaacacctataacaccattattagccgcatgggccagtggtatatgattgatatctgcctgggctataaaggcaaacgcaagattcataccgtgatctatgatagcctgaagaaactgccgtttccggtgaaaaaaatcgcgaaggactttaaactgaccgtgctgaaaggcgatattgactaccataaagaacgcccggtgggctataaaattaccccggaggaatatgcgtacatcaagaacgacatccagattattgcggaagcgctgctgattcagtttaaacagggcctggatcgtatgaccgcgggtagcgatagcctgaaaggctttaaggacattatcaccaccaagaagttcaagaaagtgtttccgaccctgagcctgggcctggataaagaagtgcgctatgcgtatcgcggtggctttacctggctgaacgatcgctttaaggaaaaggaaattggcgaaggcatggtgtttgatgtgaacagcctgtatccggcgcagatgtatagccgcctgctgccgtatggtgaaccgattgtgtttgaaggcaagtatgtgtgggatgaagattatccgctgcacattcagcatattcgctgcgaattcgaactgaaggaaggctatattccgaccattcagattaaacgcagccgcttttataaaggcaacgagtacctgaaaagcagcggcggcgaaattgcggatctgtggctgagcaacgtggatctggaactgatgaaagaacactacgatctgtacaacgtggaatatatcagcggcctgaaatttaaagcgaccaccggcctgtttaaggactttatcgacaagtggacctacattaaaaccaccagcgaaggcgcgattaaacagctggcgaaactgatgctgaacagcctgtatggcaaatttgcgagcaacccggatgttaccggcaaagtgccgtatctgaaagaaaacggcgcgctgggctttcgtttaggcgaagaggaaaccaaagatccggtgtataccccgatgggcgtgtttattaccgcgtgggcgcgctataccaccattaccgcggcgcaggcgtgttatgatcgcattatctattgcgataccgatagcattcatctgaccggcaccgaaattccggatgtgatcaaagatattgtggacccgaaaaaactgggctattgggcgcatgaaagcacctttaaacgcgcgaaatatctgcgccagaaaacctatatccaggacatctacatgaaagaggtggatggcaaactggttgaaggcagcccggatgattataccgatattaagttcagcgtgaaatgcgcgggcatgaccgataaaattaagaaggaagtgaccttcgagaactttaaagtgggctttagccgcaaaatgaaaccgaaaccggttcaggtgcctggcggtgttgttctggtggatgataccttcaccatcaagtga

SEQ ID NO: 4 - protein, 592 aa, Artificial sequence
  Synthesized Sequence
MGSSHHHHHHSSGLVPRGSHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

(19) United States
(12) Patent Application Publication
(10) Pub. No.: US 2025/0270524 A1
(43) Pub. Date: Aug. 28, 2025

(54) STABLE PHI29 DNA POLYMERASE HAVING HIGH ENZYME ACTIVITY, AND ENCODING GENE AND APPLICATION THEREOF

(71) Applicant: BGI SHENZHEN, Shenzhen (CN)

(72) Inventors: Yue Zheng, Shenzhen (CN); Xun Xu, Shenzhen (CN); Wenwei Zhang, Shenzhen (CN); Yuliang Dong, Shenzhen (CN); Yujun Zhou, Shenzhen (CN)

(21) Appl. No.: 18/008,591

(22) PCT Filed: Oct. 10, 2020
(86) PCT No.: PCT/CN2020/120099

§ 371 Date: Dec. 6, 2022

(30) Foreign Application Priority Data
Jun. 10, 2020 (CN) 202010523223.1

(51) Int. Cl. C12N 9/12; C12N 15/70; C12P 19/34

(52) U.S. Cl. CPC C12N 9/1252; C12N 15/70; C12P 19/34; C07K 2319/21; C12Y 207/07007

(57) ABSTRACT

Provided are a stable Phi29 DNA polymerase having high enzyme activity, and an encoding gene and an application thereof. The Phi29 DNA polymerase is obtained by substituting amino acid residue at at least one among the following 29 positions: position 17, position 96, position 97, position 99, position 123, position 140, position 148, position 158, position 159, position 171, position 203, position 204, position 213, position 217, position 224, position 250, position 270, position 309, position 310, position 320, position 344, position 345, position 347, position 369, position 402, position 416, position 509, position 515 and position 524 of a DNA polymerase shown in SEQ ID NO: 2.

### FIELD

The present disclosure relates to the field of biological technology, and specifically relates to a stable phi29 DNA polymerase having high enzyme activity, and an encoding gene and application thereof.

### BACKGROUND

Phi29 DNA polymerase, a thermophilic DNA polymerase cloned from Bacillus subtilis phage Phi29, is obtained by purification and isolation for mulitiple times after expressed in the Escherichia coli (E. coli) via gene recombination technology. The phi29 DNA polymerase is widely used in different isothermal amplification due to its specific strand displacement activity, high fidelity and processivity, such as rolling cycle amplification (RCA), multiple displacement amplification (MDA), loop-mediated isothermal amplification (LAMP) and so on. On the DNB-SEQ series of sequencing platforms, the phi29 DNA polymerase is mainly used in applications of DNB making and a second strand amplification. However, poor stability of wide-type Phi29 DNA polymerase and commercial phi29 DNA polymerase cannot meet requirements for presenting as a kit, for example, the wide-type Phi29 DNA polymerase is stable for less than one year and has a low reaction rate, thus exhibiting a poor sequencing quality when used for DNB-SEQ. Accordingly, it is of great significance to improve the stability and/or enzyme activity of the phi29 DNA polymerase.

### SUMMARY

The present disclosure aims at providing a phi29 DNA polymerase with an improved stability and/or enzyme activity (e.g., specific enzyme activity).

In a first aspect, the present disclosure provides in embodiments a protein which may be C1) or C2), wherein
C1) is a protein having DNA polymerase activity obtained by substituting an amino acid residue at at least one of the following 29 positions of a phi29 DNA polymerase: position 17, position 96, position 97, position 99, position 123, position 140, position 148, position 158, position 159, position 171, position 203, position 204, position 213, position 217, position 224, position 250, position 270, position 309, position 310, position 320, position 344, position 345, position 347, position 369, position 402, position 416, position 509, position 515 and position 524;
C2) is a fusion protein obtained by attaching a tag to the N-terminus or/and C-terminus of the protein of C1),
wherein an amino acid sequence of the phi29 DNA polymerase is set forth in SEQ ID NO: 2.

In the above proteins, T at position 17 may be substituted with P, R at position 96 may be substituted with E, N, S, A, G or K; M at position 97 may be substituted with Y, S, R, Q, G, C, P, V, W, N, D, E or T; Q at position 99 may be substituted with V, E or T; L at position 123 may be substituted with Y, A, C, Q, M, N or P; T at position 140 may be substituted with K or H; Y at position 148 may be substituted with P or E; I at position 158 may be substituted with P; T at position 159 may be substituted with A; Q at position 171 may be substituted with E or K; T at position 203 may be substituted with E; T at position 204 may be substituted with K; T at position 213 may be substituted with K; G at position 217 may be substituted with K; Y at position 224 may be substituted with E or K; V at position 250 may be substituted with I; V at position 270 may be substituted with R; F at position 309 may be substituted with S; Y at position 310 may be substituted with N or G; G at position 320 may be substituted with H, E, D or C; N at position 344 may be substituted with R or K, V at position 345 may be substituted with E; Y at position 347 may be substituted with G; Y at position 369 may be substituted with D or N; K at position 402 may be substituted with L; L at position 416 may be substituted with A, V at position 509 may be substituted with E; E at position 515 may be substituted with W, T, S, R, N, I, F, H, Q or T; I at position 524 may be substituted with V.

Any one of the proteins as described above exhibits higher stability and/or specific enzyme activity than that of the phi29 DNA polymerase.

Specifically, any one of the proteins as described above may be a recombinant phi29 DNA polymerase with T213K/L416A/V509E mutations, a recombinant phi29 DNA polymerase with M97T/Y224K/E515S mutations or a recombinant phi29 DNA polymerase with R96S/L123P/Y224K/L416A/E515S mutations.

The recombinant phi29 DNA polymerase with T213K/L416A/V509E mutations may be a protein obtained by substituting T at position 213 with K, L at position 416 with A and V at position 509 with E, from the N-terminus of SEQ ID NO: 2.

The recombinant phi29 DNA polymerase with M97T/Y224K/E515S mutations may be a protein obtained by substituting M at position 97 with T, Y at position 224 with K and E at position 515 with S, from the N-terminus of SEQ ID NO: 2.

The recombinant phi29 DNA polymerase with R96S/L123P/Y224K/L416A/E515S mutations may be a protein obtained by substituting R at position 96 with S, L at position 123 with P, Y at position 224 with K, L at position 416 with A and E at position 515 with S, from the N-terminus of SEQ ID NO: 2.

Any one of the proteins as described above may be any one of proteins a1 to a70.

Protein a1 is a protein obtained by substituting T at position 17 with P from the N-terminus of SEQ ID NO: 2; protein a2 is a protein obtained by substituting M at position 97 with Y from the N-terminus of SEQ ID NO: 2; protein a3 is a protein obtained by substituting M at position 97 with S from the N-terminus of SEQ ID NO: 2; protein a4 is a protein obtained by substituting M at position 97 with R from the N-terminus of SEQ ID NO: 2; protein a5 is a protein obtained by substituting M at position 97 with Q from the N-terminus of SEQ ID NO: 2; protein a6 is a protein obtained by substituting M at position 97 with G from the N-terminus of SEQ ID NO: 2; protein a7 is a protein obtained by substituting L at position 123 with Y from the N-terminus of SEQ ID NO: 2; protein a8 is a protein obtained by substituting T at position 140 with K from the N-terminus of SEQ ID NO: 2; protein a9 is a protein obtained by substituting T at position 140 with H from the N-terminus of SEQ ID NO: 2; protein a10 is a protein obtained by substituting Y at position 148 with P from the N-terminus of SEQ ID NO: 2; protein all is a protein obtained by substituting Y at position 148 with E from the N-terminus of SEQ ID NO: 2, protein a12 is a protein obtained by substituting I at position 158 with P from the N-terminus of SEQ ID NO: 2; protein a13 is a protein obtained by substituting T at position 159 with A from the N-terminus of SEQ ID NO: 2; protein a14 is a protein obtained by substituting T at position 203 with E from the N-terminus of SEQ ID NO: 2; protein a15 is a protein obtained by substituting Y at position 224 with E from the N-terminus of SEQ ID NO: 2; protein a16 is a protein obtained by substituting F at position 309 with S from the N-terminus of SEQ ID NO: 2; protein a17 is a protein obtained by substituting Y at position 310 with N from the N-terminus of SEQ ID NO: 2; protein a18 is a protein obtained by substituting Y at position 310 with G from the N-terminus of SEQ ID NO: 2; protein a19 is a protein obtained by substituting G at position 320 with H from the N-terminus of SEQ ID NO. 2; protein a20 is a protein obtained by substituting G at position 320 with E from the N-terminus of SEQ ID NO: 2; protein a21 is a protein obtained by substituting G at position 320 with D from the N-terminus of SEQ ID NO: 2; protein a22 is a protein obtained by substituting G at position 320 with C from the N-terminus of SEQ ID NO: 2; protein a23 is a protein obtained by substituting N at position 344 with R from the N-terminus of SEQ ID NO: 2; protein a24 is a protein obtained by substituting V at position 345 with E from the N-terminus of SEQ ID NO: 2; protein a25 is a protein obtained by substituting Y at position 347 with G from the N-terminus of SEQ ID NO: 2; protein a26 is a protein obtained by substituting V at position 509 with E from the N-terminus of SEQ ID NO: 2; protein a27 is a protein obtained by substituting E at position 515 with W from the N-terminus of SEQ ID NO: 2; protein a28 is a protein obtained by substituting E at position 515 with T from the N-terminus of SEQ ID NO: 2, protein a29 is a protein obtained by substituting E at position 515 with S from the N-terminus of SEQ ID NO: 2; protein a30 is a protein obtained by substituting E at position 515 with R from the N-terminus of SEQ ID NO: 2, protein a31 is a protein obtained by substituting E at position 515 with N from the N-terminus of SEQ ID NO: 2; protein a32 is a protein obtained by substituting E at position 515 with I from the N-terminus of SEQ ID NO: 2; protein a33 is a protein obtained by substituting E at position 515 with F from the N-terminus of SEQ ID NO: 2; protein a34 is a protein obtained by substituting R at position 96 with E from the N-terminus of SEQ ID NO: 2; protein a35 is a protein obtained by substituting R at position 96 with N from the N-terminus of SEQ ID NO: 2; protein a36 is a protein obtained by substituting R at position 96 with S from the N-terminus of SEQ ID NO: 2; protein a37 is a protein obtained by substituting R at position 96 with A from the N-terminus of SEQ ID NO: 2; protein a38 is a protein obtained by substituting R at position 96 with G from the N-terminus of SEQ ID NO: 2; protein a39 is a protein obtained by substituting R at position 96 with K from the N-terminus of SEQ ID NO: 2; protein a40 is a protein obtained by substituting M at position 97 with C from the N-terminus of SEQ ID NO: 2; protein a41 is a protein obtained by substituting M at position 97 with P from the N-terminus of SEQ ID NO: 2; protein a42 is a protein obtained by substituting M at position 97 with V from the N-terminus of SEQ ID NO: 2; protein a43 is a protein obtained by substituting M at position 97 with W from the N-terminus of SEQ ID NO: 2; protein a44 is a protein obtained by substituting M at position 97 with N from the N-terminus of SEQ ID NO: 2; protein a45 is a protein obtained by substituting M at position 97 with D from the N-terminus of SEQ ID NO: 2; protein a46 is a protein obtained by substituting M at position 97 with E from the N-terminus of SEQ ID NO: 2; protein a47 is a protein obtained by substituting Q at position 99 with V from the N-terminus of SEQ ID NO: 2; protein a48 is a protein obtained by substituting Q at position 99 with E from the N-terminus of SEQ ID NO: 2; protein a49 is a protein obtained by substituting Q at position 99 with T from the N-terminus of SEQ ID NO: 2; protein a50 is a protein obtained by substituting L at position 123 with A from the N-terminus of SEQ ID NO: 2; protein a51 is a protein obtained by substituting L at position 123 with C from the N-terminus of SEQ ID NO: 2; protein a52 is a protein obtained by substituting L at position 123 with Q from the N-terminus of SEQ ID NO: 2; protein a53 is a protein obtained by substituting L at position 123 with M from the N-terminus of SEQ ID NO: 2; protein a54 is a protein obtained by substituting L at position 123 with N from the N-terminus of SEQ ID NO: 2; protein a55 is a protein obtained by substituting Q at position 171 with E from the N-terminus of SEQ ID NO: 2; protein a56 is a protein obtained by substituting Q at position 171 with K from the N-terminus of SEQ ID NO: 2; protein a57 is a protein obtained by substituting T at position 204 with K from the N-terminus of SEQ ID NO: 2; protein a58 is a protein obtained by substituting T at position 213 with K from the N-terminus of SEQ ID NO: 2; protein a59 is a protein obtained by substituting G at position 217 with K from the N-terminus of SEQ ID NO: 2; protein a60 is a protein obtained by substituting V at position 250 with I from the N-terminus of SEQ ID NO: 2; protein a61 is a protein obtained by substituting V at position 270 with R from the N-terminus of SEQ ID NO: 2; protein a62 is a protein obtained by substituting N at position 344 with K from the N-terminus of SEQ ID NO: 2; protein a63 is a protein obtained by substituting Y at position 369 with D from the N-terminus of SEQ ID NO: 2; protein a64 is a protein obtained by substituting Y at position 369 with N from the N-terminus of SEQ ID NO: 2; protein a65 is a protein obtained by substituting K at position 402 with L from the N-terminus of SEQ ID NO: 2; protein a66 is a protein obtained by substituting L at position 416 with A from the N-terminus of SEQ ID NO: 2; protein a67 is a protein obtained by substituting E at position 515 with H from the N-terminus of SEQ ID NO: 2; protein a68 is a protein obtained by substituting E at position 515 with Q from the N-terminus of SEQ ID NO: 2; protein a69 is a protein obtained by substituting E at position 515 with T from the N-terminus of SEQ ID NO: 2; protein a70 is a protein obtained by substituting I at position 524 with V from the N-terminus of SEQ ID NO: 2.

Any one of the proteins as described above may be any one of proteins b1 to b70.

Protein b1 is a protein obtained by substituting T at position 37 with P from the N-terminus of SEQ ID NO: 4; protein b2 is a protein obtained by substituting M at position 117 with Y from the N-terminus of SEQ ID NO: 4; protein b3 is a protein obtained by substituting M at position 117 with S from the N-terminus of SEQ ID NO: 4; protein b4 is a protein obtained by substituting M at position 117 with R from the N-terminus of SEQ ID NO: 4; protein b5 is a protein obtained by substituting M at position 117 with Q from the N-terminus of SEQ ID NO: 4; protein b6 is a protein obtained by substituting M at position 117 with G from the N-terminus of SEQ ID NO: 4; protein b7 is a protein obtained by substituting L at position 143 with Y from the N-terminus of SEQ ID NO: 4; protein b8 is a protein obtained by substituting T at position 160 with K from the N-terminus of SEQ ID NO: 4; protein b9 is a protein obtained by substituting T at position 160 with H from the N-terminus of SEQ ID NO. 4; protein b10 is a protein obtained by substituting Y at position 168 with P from the N-terminus of SEQ ID NO: 4; protein b11 is a protein obtained by substituting Y at position 168 with E from the N-terminus of SEQ ID NO: 4; protein b12 is a protein obtained by substituting I at position 178 with P from the N-terminus of SEQ ID NO: 4; protein b13 is a protein obtained by substituting T at position 179 with A from the N-terminus of SEQ ID NO: 4; protein b14 is a protein obtained by substituting T at position 223 with E from the N-terminus of SEQ ID NO: 4; protein b15 is a protein obtained by substituting Y at position 244 with E from the N-terminus of SEQ ID NO: 4; protein b16 is a protein obtained by substituting F at position 329 with S from the N-terminus of SEQ ID NO: 4; protein b17 is a protein obtained by substituting Y at position 330 with N from the N-terminus of SEQ ID NO: 4; protein b18 is a protein obtained by substituting Y at position 330 with G from the N-terminus of SEQ ID NO: 4; protein b19 is a protein obtained by substituting G at position 340 with H from the N-terminus of SEQ ID NO: 4; protein b20 is a protein obtained by substituting G at position 340 with E from the N-terminus of SEQ ID NO: 4; protein b21 is a protein obtained by substituting G at position 340 with D from the N-terminus of SEQ ID NO: 4; protein b22 is a protein obtained by substituting G at position 340 with C from the N-terminus of SEQ ID NO: 4; protein b23 is a protein obtained by substituting N at position 364 with R from the N-terminus of SEQ ID NO: 4; protein b24 is a protein obtained by substituting V at position 365 with E from the N-terminus of SEQ ID NO: 4; protein b25 is a protein obtained by substituting Y at position 367 with G from the N-terminus of SEQ ID NO: 4; protein b26 is a protein obtained by substituting V at position 529 with E from the N-terminus of SEQ ID NO: 4; protein b27 is a protein obtained by substituting E at position 535 with W from the N-terminus of SEQ ID NO: 4; protein b28 is a protein obtained by substituting E at position 535 with T from the N-terminus of SEQ ID NO: 4; protein b29 is a protein obtained by substituting E at position 535 with S from the N-terminus of SEQ ID NO: 4; protein b30 is a protein obtained by substituting E at position 535 with R from the N-terminus of SEQ ID NO: 4; protein b31 is a protein obtained by substituting E at position 535 with N from the N-terminus of SEQ ID NO: 4; protein b32 is a protein obtained by substituting E at position 535 with I from the N-terminus of SEQ ID NO: 4; protein b33 is a protein obtained by substituting E at position 535 with F from the N-terminus of SEQ ID NO: 4; protein b34 is a protein obtained by substituting R at position 116 with E from the N-terminus of SEQ ID NO: 4; protein b35 is a protein obtained by substituting R at position 116 with N from the N-terminus of SEQ ID NO: 4; protein b36 is a protein obtained by substituting R at position 116 with S from the N-terminus of SEQ ID NO: 4; protein b37 is a protein obtained by substituting R at position 116 with A from the N-terminus of SEQ ID NO: 4; protein b38 is a protein obtained by substituting R at position 116 with G from the N-terminus of SEQ ID NO: 4; protein b39 is a protein obtained by substituting R at position 116 with K from the N-terminus of SEQ ID NO: 4; protein b40 is a protein obtained by substituting M at position 117 with C from the N-terminus of SEQ ID NO: 4; protein b41 is a protein obtained by substituting M at position 117 with P from the N-terminus of SEQ ID NO: 4; protein b42 is a protein obtained by substituting M at position 117 with V from the N-terminus of SEQ ID NO: 4; protein b43 is a protein obtained by substituting M at position 117 with W from the N-terminus of SEQ ID NO: 4; protein b44 is a protein obtained by substituting M at position 117 with N from the N-terminus of SEQ ID NO: 4; protein b45 is a protein obtained by substituting M at position 117 with D from the N-terminus of SEQ ID NO: 4; protein b46 is a protein obtained by substituting M at position 117 with E from the N-terminus of SEQ ID NO: 4; protein b47 is a protein obtained by substituting Q at position 119 with V from the N-terminus of SEQ ID NO: 4; protein b48 is a protein obtained by substituting Q at position 119 with E from the N-terminus of SEQ ID NO: 4; protein b49 is a protein obtained by substituting Q at position 119 with T from the N-terminus of SEQ ID NO: 4; protein b50 is a protein obtained by substituting L at position 143 with A from the N-terminus of SEQ ID NO: 4; protein b51 is a protein obtained by substituting L at position 143 with C from the N-terminus of SEQ ID NO: 4; protein b52 is a protein obtained by substituting L at position 143 with Q from the N-terminus of SEQ ID NO: 4; protein b53 is a protein obtained by substituting L at position 143 with M from the N-terminus of SEQ ID NO: 4; protein b54 is a protein obtained by substituting L at position 143 with N from the N-terminus of SEQ ID NO: 4; protein b55 is a protein obtained by substituting Q at position 191 with E from the N-terminus of SEQ ID NO: 4; protein b56 is a protein obtained by substituting Q at position 191 with K from the N-terminus of SEQ ID NO: 4; protein b57 is a protein obtained by substituting T at position 224 with K from the N-terminus of SEQ ID NO: 4; protein b58 is a protein obtained by substituting T at position 233 with K from the N-terminus of SEQ ID NO: 4; protein b59 is a protein obtained by substituting G at position 237 with K from the N-terminus of SEQ ID NO: 4; protein b60 is a protein obtained by substituting V at position 270 with I from the N-terminus of SEQ ID NO: 4; protein b61 is a protein obtained by substituting V at position 290 with R from the N-terminus of SEQ ID NO: 4; protein b62 is a protein obtained by substituting N at position 364 with K from the N-terminus of SEQ ID NO: 4; protein b63 is a protein obtained by substituting Y at position 389 with D from the N-terminus of SEQ ID NO: 4; protein b64 is a protein obtained by substituting Y at position 389 with N from the N-terminus of SEQ ID NO: 4; protein b65 is a protein obtained by substituting K at position 422 with L from the N-terminus of SEQ ID NO: 4; protein b66 is a protein obtained by substituting L at position 436 with A from the N-terminus of SEQ ID NO: 4; protein b67 is a protein obtained by substituting E at position 535 with H from the N-terminus of SEQ ID NO: 4; protein b68 is a protein obtained by substituting E at position 535 with Q from the N-terminus of SEQ ID NO: 4; protein b69 is a protein obtained by substituting E at position 535 with T from the N-terminus of SEQ ID NO: 4; protein b70 is a protein obtained by substituting I at position 544 with V from the N-terminus of SEQ ID NO: 4.

A nucleic acid molecule encoding a protein of any one of the proteins as described above is also drawn into the protect scope of the present disclosure.

An expression cassette, a recombinant vector, a recombinant microorganism or a transgenic cell line comprising the nucleic acid molecule is also drawn into the protect scope of the present disclosure.

The recombinant vector may be a recombinant plasmid obtained by inserting the nucleic acid molecule into an expression vector or a cloning vector. Specifically, the expression vector may be a pET28a (+) vector.

Specifically, the recombinant vector may be a recombinant pET28a-T17P vector, a recombinant pET28a-M97Y vector, a recombinant pET28a-M97S vector, a recombinant pET28a-M97R vector, a recombinant pET28a-M97Q vector, a recombinant pET28a-M97G vector, a recombinant pET28a-L123Y vector, a recombinant pET28a-T140K vector, a recombinant pET28a-T140H vector, a recombinant pET28a-Y148P vector, a recombinant pET28a-Y148E vector, a recombinant pET28a-1158P vector, a recombinant pET28a-T159A vector, a recombinant pET28a-T203E vector, a recombinant pET28a-Y224E vector, a recombinant pET28a-F309S vector, a recombinant pET28a-Y310N vector, a recombinant pET28a-Y310G vector, a recombinant a recombinant pET28a-G320H vector, pET28a-G320E vector, a recombinant pET28a-G320D vector, a recombinant pET28a-G320C vector, a recombinant pET28a-N344R vector, a recombinant pET28a-V345E vector, a recombinant pET28a-Y347G vector, a recombinant vector, vector, a recombinant pET28a-V509E a recombinant pET28a-E515W pET28a-E515T vector, a recombinant pET28a-E515S vector, a recombinant pET28a-E515R vector, a recombinant pET28a-E515N vector, a recombinant pET28a-E515 I vector, a recombinant pET28a-E515F vector, a recombinant pET28a-R96E vector, a recombinant pET28a-R96N vector, a recombinant pET28a-R96S vector, a recombinant pET28a-R96A vector, a recombinant pET28a-R96G vector, a recombinant pET28a-R96K vector, a recombinant pET28a-M97C vector, a recombinant pET28a-M97P vector, a recombinant pET28a-M97V vector, a recombinant pET28a-M97W vector, a recombinant pET28a-M97N vector, a recombinant pET28a-M97D vector, a recombinant pET28a-M97E vector, a recombinant pET28a-Q99V vector, a recombinant pET28a-Q99E vector, a recombinant pET28a-Q99T vector, a recombinant pET28a-L123A vector, a recombinant pET28a-L123C vector, a recombinant pET28a-L123Q vector, a recombinant pET28a-L123M vector, a recombinant pET28a-L123N vector, a recombinant pET28a-Q171E vector, a recombinant pET28a-Q171K vector, a recombinant pET28a-T204K vector, a recombinant pET28a-T213K vector, a recombinant pET28a-G217K vector, a recombinant pET28a-V250I vector, a recombinant pET28a-V270R vector, a recombinant pET28a-N344K vector, a recombinant pET28a-Y369D vector, a recombinant pET28a-Y369N vector, a recombinant pET28a-K402L vector, a recombinant pET28a-L416A vector, a recombinant pET28a-E515H vector, a recombinant pET28a-E515Q vector, a recombinant pET28a-E515T or a recombinant pET28a-1524V, mentioned in embodiments of the present disclosure.

The recombinant microorganism is a recombinant bacterium obtained by introducing the recombinant vector into an initial microorganism.

The initial microorganism may be E. coli.

Specifically, the E. coli may be E. coli BL21 (DE3).

Use of any one of the proteins or the nucleic acid molecules as described above in preparation of a DNA polymerase is also drawn into the protect scope of the present disclosure.

In the above use, the recombinant DNA polymerase exhibits higher stability and/or specific enzyme activity than that of a phi29 DNA polymerase.

Use of any one of the proteins or the nucleic acid molecules as described above in PCR amplification or sequencing is also drawn into the protect scope of the present disclosure.

In the above use, the PCR amplification may be second strand amplification, single cell amplification and/or plasmid amplification, and the sequencing may be DNB SEQ sequencing.

Use of any one of the proteins or the nucleic acid molecules as described above in preparation of a product for sequencing is also drawn into the protect scope of the present disclosure.

In the above use, the product may be a kit.

The inventors of the present disclosure have conducted site-directed mutagenesis on the existing phi29 DNA polymerase through a large number of experiments, and further used DNA shuffling and a combined-mutation construction method to construct a combined mutant, and prepared 73 recombinant phi29 DNA polymerases with significantly improved stability and/or specific enzyme activity. These recombinant phi29 DNA polymerases not only have improved thermal stability, but also exhibit increased polymerization activity and processivity. When the recombinant phi29 DNA polymerases prepared in the present disclosure are used in amplification or sequencing, DNA can be efficiently and continuously synthesized, and the reaction efficiency is high. The present disclosure has important application value.

### BRIEF DESCRIPTION OF THE DRAWINGS

FIG. 1 is a structure diagram showing a pET28a (+) vector.

### DETAILED DESCRIPTION OF THE DISCLOSURE

The following examples are for better understanding of the present disclosure rather than limiting.

Unless otherwise specified, the experimental methods in the following examples are conventional methods.

The test materials used in the following examples are purchased from conventional biochemical reagent companies, unless otherwise specified.

The quantitative experiments in the following examples are all set up in triplicate, with averaged results.

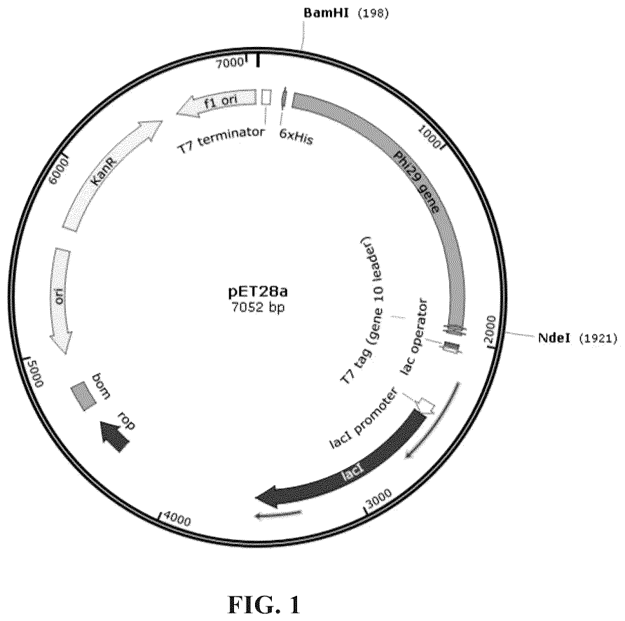

The pET28a (+) vector is purchased from Novagen Company, a structure diagram of which is shown in FIG. 1.

Affinity solution A was an aqueous solution containing 20 mM of Tris-HCl, 500 mM of NaCl, 20 mM of Imidazole and 62.5 g/L of Glycerol, pH value of which was 7.9.

Recombinant phi29 DNA polymerases in Examples 1, 2 and 3 were phi29 DNA polymerases with a single mutation, while recombinant phi29 DNA polymerases in Examples 4 were phi29 DNA polymerases with combined mutations.

### Example 1: Preparation of a Crude Recombinant Phi29 DNA Polymerase

### 1.1 Construction of a Recombinant Plasmid pET28a-WT

A short DNA fragment between the sequences recognized by restriction enzymes of NdeI and BamHI in pET28a (+) was replaced by a double-stranded DNA molecule as shown in SEQ ID NO: 1, and other sequences were unchanged, thereby obtaining the recombinant plasmid pET28a-WT.

The double-stranded DNA molecule as shown in SEQ ID NO: 1 was a coding gene for the Phi29 DNA polymerase, which encodes the Phi29 DNA polymerase with an amino acid sequence as shown in SEQ ID NO: 2.

The recombinant plasmid pET28a-WT was sequenced. The sequencing results showed that in the recombinant plasmid pET28a-WT, the double-stranded DNA molecule shown in SEQ ID NO: 1 was fused with a coding sequence of a His-tag composed of 6 histidine residues on the vector backbone, forming a fusion gene as shown in SEQ ID NO: 3 that expressed the recombinant Phi29 DNA polymerase as shown in SEQ ID NO: 4, which was named as fusion protein 1, where the fusion protein 1 with the His-tag.

### 1.2 Site-Directed Mutagenesis of the Coding Gene of the Phi29 DNA Polymerase

1.2.1 A PCR reaction system for the site-directed mutagenesis was prepared. The PCR reaction system for the site-directed mutagenesis was in a volume of 25 μl, including 2.5 μl of 10×Pfu Reaction Buffer with Mg²⁺; 2 μl of dNTP Mix, where the concentrations of dATP, dTTP, dGTP and dCTP each were 2.5 mM; 25 ng of the recombinant plasmid pET28a-WT; 0.5 μl of Pfu DNA Polymerase; and a primer for introducing a mutation.

Pfu DNA Polymerase is purchased from ThermoFisher Company with Cat. No. EP0501. 10×Pfu Reaction Buffer with Mg²⁺ is a component of Pfu DNA Polymerase.

The primers for introducing a mutation are shown in Table 1.

TABLE 1

| mutation | nucleotide sequence of forward | nucleotide sequence of reverse |
|---|---|---|
| site | primer (5′-3′) | primer (5′-3′) |
| T17P | GACTTTGAAACCACCCCGAAAGT | GCAATCTTCCACTTTCGGGGTGGT |
| | GGAAGATTGC | TTCAAAGTC |
| M97Y | GATATCAATCATATACCACTGGC | ACCTATAACACCATTATTAGCCGC |
| | CATAGCGGCTAATAATGGTGTTA | TATGGCCAGTGGTATATGATTGAT |
| | TAGGT | ATC |
| M97S | CATATACCACTGGCCGCTGCGGC | CCTATAACACCATTATTAGCCGCA |
| | TAATAATGGTGTTATAGG | GCGGCCAGTGGTATATG |
| M97R | TACCACTGGCCCCTGCGGCTAAT | CTATAACACCATTATTAGCCGCAG |
| | AATGGTGTTATAG | GGGCCAGTGGTA |
| M97Q | CAATCATATACCACTGGCCCTGG | ATAACACCATTATTAGCCGCCAGG |
| | CGGCTAATAATGGTGTTAT | GCCAGTGGTATATGATTG |
| M97G | CAATCATATACCACTGGCCCCCG | ATAACACCATTATTAGCCGCGGGG |
| | CGGCTAATAATGGTGTTAT | GCCAGTGGTATATGATTG |
| L123Y | CACCGGAAACGGCAGTTTCTTAT | CATACCGTGATCTATGATAGCTAT |
| | AGCTATCATAGATCACGGTATG | AAGAAACTGCCGTTTCCGGTG |
| T140K | ATATCGCCTTTCAGCACCTTCAG | TCGCGAAGGACTTTAAACTGAAGG |
| | TTTAAAGTCCTTCGCGA | TGCTGAAAGGCGATAT |
| T140H | AATATCGCCTTTCAGCACGTGCA | CGCGAAGGACTTTAAACTGCACGT |
| | GTTTAAAGTCCTTCGCG | GCTGAAAGGCGATATT |
| Y148P | AAAGGCGATATTGACCCGCATA | CGGGCGTTCTTTATGCGGGTCAAT |
| | AAGAACGCCCG | ATCGCCTTT |
| Y148E | AAAGGCGATATTGACGAACATA | CGGGCGTTCTTTATGTTCGTCAAT |
| | AAGAACGCCCG | ATCGCCTTT |
| I158P | CCGGTGGGCTATAAACCGACCCC | ATATTCCTCCGGGGTCGGTTTATA |
| | GGAGGAATAT | GCCCACCGG |
| T159A | GTGGGCTATAAAATTGCGCCGGA | CGCATATTCCTCCGGCGCAATTTT |
| | GGAATATGCG | ATAGCCCAC |
| T203E | GAAACACTTTCTTGAACTTCTTG | GCCTGAAAGGCTTTAAGGACATTA |
| | GTCTCGATAATGTCCTTAAAGCC | TCGAGACCAAGAAGTTCAAGAAA |
| | TTTCAGGC | GTGTTTC |
| Y224E | AGCCACCGCGATACGCCTCGCGC | GGATAAAGAAGTGCGCGAGGCGT |
| | ACTTCTTTATCC | ATCGCGGTGGCT |
| F309S | ATTAAACGCAGCCGCAGCTATAA | CTCGTTGCCTTTATAGCTGCGGCT |
| | AGGCAACGAG | GCGTTTAAT |
| Y310N | AAACGCAGCCGCTTTAATAAAG | GTACTCGTTGCCTTTATTAAAGCG |
| | GCAACGAGTAC | GCTGCGTTT |
| Y310G | AAACGCAGCCGCTTTGGTAAAG | GTACTCGTTGCCTTTACCAAAGCG |
| | GCAACGAGTAC | GCTGCGTTT |
| G320H | TACCTGAAAAGCAGCCATGGCG | ATCCGCAATTTCGCCATGGCTGCT |
| | AAATTGCGGAT | TTTCAGGTA |
| G320E | TACCTGAAAAGCAGCGAAGGCG | ATCCGCAATTTCGCCTTCGCTGCTT |
| | AAATTGCGGAT | TTCAGGTA |
| G320D | TACCTGAAAAGCAGCGATGGCG | ATCCGCAATTTCGCCATCGCTGCT |
| | AAATTGCGGAT | TTTCAGGTA |
| G320C | TACCTGAAAAGCAGCTGTGGCG | ATCCGCAATTTCGCCACAGCTGCT |
| | AAATTGOGGAT | TTTCAGGTA |
| N344R | CACTACGATCTGTACCGTGTGGA | GCTGATATATTCCACACGGTACAG |
| | ATATATCAGC | ATCGTAGTG |
| V345E | TACGATCTGTACAACGAAGAATA | GCCGCTGATATATTCTTCGTTGTA |
| | TATCAGCGGC | CAGATCGTA |
| Y347G | TTTAAATTTCAGGCCGCTGATAC | CTACGATCTGTACAACGTGGAAGG |
| | CTTCCACGTTGTACAGATCGTAG | TATCAGCGGCCTGAAATTTAAA |
| V509E | ATCTACATGAAAGAGGAAGATG | AACCAGTTTGCCATCTTCCTCTTTC |
| | GCAAACTGGTT | ATGTAGAT |
| ES15W | CATCCGGGCTGCCCCAAACCAGT | GAAAGAGGTGGATGGCAAACTGG |
| | TTGCCATCCACCTCTTTC | TTTGGGGCAGCCCGGATG |
| E515T | GAAAGAGGTGGATGGCAAACTG | CATCCGGGCTGCCCGTAACCAGTT |
| | GTTACGGGCAGCCCGGATG | TGCCATCCACCTCTTTC |
| E515S | GAGGTGGATGGCAAACTGGTTTC | CCGGGCTGCCTGAAACCAGTTTGC |
| | AGGCAGCCCGG | CATCCACCTC |
| E515R | CCGGGCTGCCTCTAACCAGTTTG | GAGGTGGATGGCAAACTGGTTAG |
| | CCATCCACCTC | AGGCAGCCCGG |
| E515N | CCGGGCTGCCATTAACCAGTTTG | GGTGGATGGCAAACTGGTTAATGG |
| | CCATCCACC | CAGCCCGG |
| E515I | CCGGGCTGCCTATAACCAGTTTG | GAGGTGGATGGCAAACTGGTTATA |
| | CCATCCACCTC | GGCAGCCCGG |
| E515F | CATCCGGGCTGCCGAAAACCAGT | GAAAGAGGTGGATGGCAAACTGG |
| | TTGCCATCCACCTCTTTC | TTTTCGGCAGCCCGGATG |
| R96E | CATATACCACTGGCCCATCTCGC | CGAACACCTATAACACCATTATTA |
| | TAATAATGGTGTTATAGGTGTTC | GCGAGATGGGCCAGTGGTATATG |
| | G | |
| R96N | AACACCATTATTAGCAATATGGG | ATACCACTGGCCCATATTGCTAAT |
| | CCAGTGGTAT | AATGGTGTT |
| R96A | AACACCATTATTAGCGCGATGGG | ATACCACTGGCCCATCGCGCTAAT |
| | CCAGTGGTAT | AATGGTGTT |
| R96G | AACACCATTATTAGCGGTATGGG | ATACCACTGGCCCATACCGCTAAT |
| | CCAGTGGTAT | AATGGTGTT |
| R96K | CATATACCACTGGCCCATCTTGC | CGAACACCTATAACACCATTATTA |
| | TAATAATGGTGTTATAGGTGTTC | GCAAGATGGGCCAGTGGTATATG |
| | G | |
| M97V | CATATACCACTGGCCCACGCGGC | ACACCATTATTAGCCGCGTGGGCC |
| | TAATAATGGTGT | AGTGGTATATG |
| M97C | ATATCAATCATATACCACTGGCC | CCTATAACACCATTATTAGCCGCT |
| | GCAGCGGCTAATAATGGTGTTAT | GCGGCCAGTGGTATATGATTGATA |
| | AGG | T |
| M97P | ATAACACCATTATTAGCCGCCCG | CAATCATATACCACTGGCCCGGGC |
| | GGCCAGTGGTATATGATTG | GGCTAATAATGGTGTTAT |
| M97W | CAATCATATACCACTGGCCCCAG | ATAACACCATTATTAGCCGCTGGG |
| | CGGCTAATAATGGTGTTAT | GCCAGTGGTATATGATTG |
| M97N | CATATACCACTGGCCATTGCGGC | CCTATAACACCATTATTAGCCGCA |
| | TAATAATGGTGTTATAGG | ATGGCCAGTGGTATATG |
| M97D | GATATCAATCATATACCACTGGC | ACCTATAACACCATTATTAGCCGC |
| | CATCGCGGCTAATAATGGTGTTA | GATGGCCAGTGGTATATGATTGAT |
| | TAGGT | ATC |
| M97E | CAATCATATACCACTGGCCCTCG | ATAACACCATTATTAGCCGCGAGG |
| | CGGCTAATAATGGTGTTAT | GCCAGTGGTATATGATTG |
| M97Y | GATATCAATCATATACCACTGGC | ACCTATAACACCATTATTAGCCGC |
| | CATAGCGGCTAATAATGGTGTTA | TATGGCCAGTGGTATATGATTGAT |
| | TAGGT | ATC |
| M97R | TACCACTGGCCCCTGCGGCTAAT | CTATAACACCATTATTAGCCGCAG |
| | AATGGTGTTATAG | GGGCCAGTGGTA |
| M97S | CATATACCACTGGCCGCTGCGGC | CCTATAACACCATTATTAGCCGCA |
| | TAATAATGGTGTTATAGG | GCGGCCAGTGGTATATG |
| Q99V | CAGATATCAATCATATACCACAC | CCATTATTAGCCGCATGGGCGTGT |
| | GCCCATGCGGCTAATAATGG | GGTATATGATTGATATCTG |
| Q99E | GATATCAATCATATACCACTCGC | CATTATTAGCCGCATGGGCGAGTG |
| | CCATGCGGCTAATAATG | GTATATGATTGATATC |
| Q99T | ATTAGCCGCATGGGCACCTGGTA | ATCAATCATATACCAGGTGCCCAT |
| | TATGATTGAT | GCGGCTAAT |
| L123A | CGGAAACGGCAGTTTCTTCGCGC | TACCGTGATCTATGATAGCGCGAA |
| | TATCATAGATCACGGTA | GAAACTGCCGTTTCCG |
| L123C | CACCGGAAACGGCAGTTTCTTGC | CATACCGTGATCTATGATAGCTGC |
| | AGCTATCATAGATCACGGTATG | AAGAAACTGCCGTTTCCGGTG |
| L123Q | GAAACGGCAGTTTCTTCTGGCTA | CGTGATCTATGATAGCCAGAAGAA |
| | TCATAGATCACG | ACTGCCGTTTC |
| L123M | GAAACGGCAGTTTCTTCATGCTA | ACCGTGATCTATGATAGCATGAAG |
| | TCATAGATCACGGT | AAACTGCCGTTTC |
| L123N | CACCGGAAACGGCAGTTTCTTAT | CATACCGTGATCTATGATAGCAAT |
| | TGCTATCATAGATCACGGTATG | AAGAAACTGCCGTTTCCGGTG |
| Q171E | ACATCAAGAACGACATCGAGAT | CGCTTCCGCAATAATCTCGATGTC |
| | TATTGCGGAAGCG | GTTCTTGATGT |
| Q171K | ATCAAGAACGACATCAAAATTAT | CGCTTCCGCAATAATTTTGATGTC |
| | TGCGGAAGCG | GTTCTTGAT |
| T204K | AAGGACATTATCACCAAGAAGA | TTTCTTGAACTTCTTCTTGGTGATA |
| | AGTTCAAGAAA | ATGTCCTT |
| T213K | AAGAAAGTGTTTCCGAAACTGA | CAGGCCCAGGCTCAGTTTCGGAAA |
| | GCCTGGGCCTG | CACTTTCTT |
| G217K | CCGACCCTGAGCCTGAAACTGGA | CACTTCTTTATCCAGTTTCAGGCTC |
| | TAAAGAAGTG | AGGGTCGG |
| V250I | GGATACAGGCTGTTTATATCAAA | AAATTGGCGAAGGCATGGTGTTTG |
| | CACCATGCCTTCGCCAATTT | ATATAAACAGCCTGTATCC |
| V270R | TATGGTGAACCGATTCGTTTTGA | ATACTTGCCTTCAAAACGAATCGG |
| | AGGCAAGTAT | TTCACCATA |
| N344K | CACTACGATCTGTACAAAGTGGA | GCTGATATATTCCACTTTGTACAG |
| | ATATATCAGC | ATCGTAGTG |
| Y369D | ATCGACAAGTGGACCGATATTAA | GCTGGTGGTTTTAATATCGGTCCA |
| | AACCACCAGC | CTTGTCGAT |
| Y369N | ATCGACAAGTGGACCAATATTAA | GCTGGTGGTTTTAATATTGGTCCA |
| | AACCACCAGC | CTTGTCGAT |
| K402L | CTTTCAGATACGGCACTAAGCCG | ACCCGGATGTTACCGGCTTAGTGC |
| | GTAACATCCGGGT | CGTATCTGAAAG |
| L416A | GCGCTGGGCTTTCGTGCGGGCGA | GGTTTCCTCTTCGCCCGCACGAAA |
| | AGAGGAAACC | GCCCAGCGC |
| E515F | CATCCGGGCTGCCGAAAACCAGT | GAAAGAGGTGGATGGCAAACTGG |
| | TTGCCATCCACCTCTTTC | TTTTCGGCAGCCCGGATG |
| E515H | CCGGGCTGCCATGAACCAGTTTG | GGTGGATGGCAAACTGGTTCATGG |
| | CCATCCACC | CAGCCCGG |
| E515Q | CCGGGCTGCCCTGAACCAGTTTG | GGTGGATGGCAAACTGGTTCAGG |
| | CCATCCACC | GCAGCCCGG |
| E515T | GAAAGAGGTGGATGGCAAACTG | CATCCGGGCTGCCCGTAACCAGTT |
| | GTTACGGGCAGCCCGGATG | TGCCATCCACCTCTTTC |
| I524V | GATGATTATACCGATGTGAAGTT | TTTCACGCTGAACTTCACATCGGT |
| | CAGCGTGAAA | ATAATCATC |

1.2.2 PCR amplification was proformed with the PCR reaction system for the site-directed mutagenesis, and a PCR amplified product was obtained.

The reaction program was set as (i) 95° C. for 3 min; (ii) 95° C. for 30 s, 53° C. for 30 s, 68° C. for 8 min, where (ii) was proformed for 19 cycles; and (iii) 4° C. for hold.

1.2.3 The PCR amplified product was disgested by DpnI enzyme and transformed into E. coli DH5a competent cells, followed by spreading on Luria-Bertani (LB) medium plates containing kanamycin and culturing at 37° C. overnight. Single clones were picked and plasmids therein were extracted.

1.2.4 The plasmids extracted at step 1.2.3 were sequenced indivadully. Based on the sequencing results, several recombinant plasmids each with a site mutation in the gene encoding the Phi29 DNA polymerase were obtained, for encoding different fusion proteins respectively (i.e. recombinant phi29 DNA polymerase).

The fusion proteins encoded by part of recombinant plasmids are shown in Table 2.

TABLE 2

| Name of recombinant | Encoded fusion | Difference with the |
|---|---|---|
| plasmid | protein | fusion protein 1 |
| recombinant plasmid | fusion protein 2 | T at position 37 was |
| pET28a-T17P | | substituted with P |
| recombinant plasmid | fusion protein 3 | M at position 117 was |
| pET28a-M97Y | | substituted with Y |
| recombinant plasmid | fusion protein 4 | M at position 117 was |
| pET28a-M97S | | substituted with S |
| recombinant plasmid | fusion protein 5 | M at position 117 was |
| pET28a-M97R | | substituted with R |
| recombinant plasmid | fusion protein 6 | M at position 117 was |
| pET28a-M97Q | | substituted with Q |
| recombinant plasmid | fusion protein 7 | M at position 117 was |
| pET28a-M97G | | substituted with G |
| recombinant plasmid | fusion protein 8 | L at position 143 was |
| pET28a-L123Y | | substituted with Y |
| recombinant plasmid | fusion protein 9 | T at position 160 was |
| pET28a-T140K | | substituted with K |
| recombinant plasmid | fusion protein 10 | T at position 160 was |
| pET28a-T140H | | substituted with H |
| recombinant plasmid | fusion protein 11 | Y at position 168 was |
| pET28a-Y148P | | substituted with P |
| recombinant plasmid | fusion protein 12 | Y at position 168 was |
| pET28a-Y148E | | substituted with E |
| recombinant plasmid | fusion protein 13 | I at position 178 was |
| pET28a-I158P | | substituted with P |
| recombinant plasmid | fusion protein 14 | T at position 179 was |
| pET28a-T159A | | substituted with A |
| recombinant plasmid | fusion protein 15 | T at position 223 was |
| pET28a-T203E | | substituted with E |
| recombinant plasmid | fusion protein 16 | Y at position 244 was |
| pET28a-Y224E | | substituted with E |
| recombinant plasmid | fusion protein 17 | F at position 329 was |
| pET28a-F309S | | substituted with S |
| recombinant plasmid | fusion protein 18 | Y at position 330 was |
| pET28a-Y310N | | substituted with N |
| recombinant plasmid | fusion protein 19 | Y at position 330 was |
| pET28a-Y310G | | substituted with G |
| recombinant plasmid | fusion protein 20 | G at position 340 was |
| pET28a-G320H | | substituted with H |
| recombinant plasmid | fusion protein 21 | G at position 340 was |
| pET28a-G320E | | substituted with E |
| recombinant plasmid | fusion protein 22 | G at position 340 was |
| pET28a-G320D | | substituted with D |
| recombinant plasmid | fusion protein 23 | G at position 340 was |
| pET28a-G320C | | substituted with C |
| recombinant plasmid | fusion protein 24 | N at position 364 was |
| pET28a-N344R | | substituted with R |
| recombinant plasmid | fusion protein 25 | V at position 365 was |
| pET28a-V345E | | substituted with E |
| recombinant plasmid | fusion protein 26 | Y at position 367 was |
| pET28a-Y347G | | substituted with G |
| recombinant plasmid | fusion protein 27 | V at position 529 was |
| pET28a-V509E | | substituted with E |
| recombinant plasmid | fusion protein 28 | E at position 535 was |
| pET28a-E515W | | substituted with W |
| recombinant plasmid | fusion protein 29 | E at position 535 was |
| pET28a-E515T | | substituted with T |
| recombinant plasmid | fusion protein 30 | E at position 535 was |
| pET28a-E515S | | substituted with S |
| recombinant plasmid | fusion protein 31 | E at position 535 was |
| pET28a-E515R | | substituted with R |
| recombinant plasmid | fusion protein 32 | E at position 535 was |
| pET28a-E515N | | substituted with N |
| recombinant plasmid | fusion protein 33 | E at position 535 was |
| pET28a-E515I | | substituted with I |
| recombinant plasmid | fusion protein 34 | E at position 535 was |
| pET28a-E515F | | substituted with F |
| recombinant plasmid | fusion protein 35 | R at position 116 was |
| pET28a-R96E | | substituted with E |
| recombinant plasmid | fusion protein 36 | R at position 116 was |
| pET28a-R96N | | substituted with N |
| recombinant plasmid | fusion protein 37 | R at position 116 was |
| pET28a-R96S | | substituted with S |
| recombinant plasmid | fusion protein 38 | R at position 116 was |
| pET28a-R96A | | substituted with A |
| recombinant plasmid | fusion protein 39 | R at position 116 was |
| pET28a-R96G | | substituted with G |
| recombinant plasmid | fusion protein 40 | R at position 116 was |
| pET28a-R96K | | substituted with K |
| recombinant plasmid | fusion protein 41 | M at position 117 was |
| pET28a-M97C | | substituted with C |
| recombinant plasmid | fusion protein 42 | M at position 117 was |
| pET28a-M97P | | substituted with P |
| recombinant plasmid | fusion protein 43 | M at position 117 was |
| pET28a-M97V | | substituted with V |
| recombinant plasmid | fusion protein 44 | M at position 117 was |
| pET28a-M97W | | substituted with W |
| recombinant plasmid | fusion protein 45 | M at position 117 was |
| pET28a-M97N | | substituted with N |
| recombinant plasmid | fusion protein 46 | M at position 117 was |
| pET28a-M97D | | substituted with D |
| recombinant plasmid | fusion protein 47 | M at position 117 was |
| pET28a-M97E | | substituted with E |
| recombinant plasmid | fusion protein 48 | Q at position 119 was |
| pET28a-Q99V | | substituted with V |
| recombinant plasmid | fusion protein 49 | Q at position 119 was |
| pET28a-Q99E | | substituted with E |
| recombinant plasmid | fusion protein 50 | Q at position 119 was |
| pET28a-Q99T | | substituted with T |
| recombinant plasmid | fusion protein 51 | L at position 143 was |
| pET28a-L123A | | substituted with A |
| recombinant plasmid | fusion protein 52 | L at position 143 was |
| pET28a-L123C | | substituted with C |
| recombinant plasmid | fusion protein 53 | L at position 143 was |
| pET28a-L123Q | | substituted with Q |
| recombinant plasmid | fusion protein 54 | L at position 143 was |
| pET28a-L123M | | substituted with M |
| recombinant plasmid | fusion protein 55 | L at position 143 was |
| pET28a-L123N | | substituted with N |
| recombinant plasmid | fusion protein 56 | Q at position 191 was |
| pET28a-Q171E | | substituted with E |
| recombinant plasmid | fusion protein 57 | Q at position 191 was |
| pET28a-Q171K | | substituted with K |
| recombinant plasmid | fusion protein 58 | T at position 224 was |
| pET28a-T204K | | substituted with K |
| recombinant plasmid | fusion protein 59 | T at position 233 was |
| pET28a-T213K | | substituted with K |
| recombinant plasmid | fusion protein 60 | G at position 237 was |
| pET28a-G217K | | substituted with K |
| recombinant plasmid | fusion protein 61 | V at position 270 was |
| pET28a-V250I | | substituted with I |
| recombinant plasmid | fusion protein 62 | V at position 290 was |
| pET28a-V270R | | substituted with R |
| recombinant plasmid | fusion protein 63 | N at position 364 was |
| pET28a-N344K | | substituted with K |
| recombinant plasmid | fusion protein 64 | Y at position 389 was |
| pET28a-Y369D | | substituted with D |
| recombinant plasmid | fusion protein 65 | Y at position 389 was |
| pET28a-Y369N | | substituted with N |
| recombinant plasmid | fusion protein 66 | K at position 422 was |
| pET28a-K402L | | substituted with L |
| recombinant plasmid | fusion protein 67 | L at position 436 was |
| pET28a-L416A | | substituted with A |
| recombinant plasmid | fusion protein 68 | E at position 535 was |
| pET28a-E515H | | substituted with H |
| recombinant plasmid | fusion protein 69 | E at position 535 was |
| pET28a-E515Q | | substituted with Q |
| recombinant plasmid | fusion protein 70 | E at position 535 was |
| pET28a-E515T | | substituted with T |
| recombinant plasmid | fusion protein 71 | I at position 544 was |
| pET28a-I524V | | substituted with V |

### 1.3 Preparation of the Crude Recombinant Phi29 DNA Polymerase

A method for the preparing the crude recombinant phi29 DNA polymerase was as follows.

1.3.1 The recombinant plasmid pET28a-WT was transformed into E. coli BL21 (DE3) to obtain a recombinant bacterium named as BL21 (DE3)-WT.

1.3.2 Single clones of BL21 (DE3)-WT were picked and transferred into 5 ml LB fluid medium containing 50 μg/ml kanamycin, and cultured under shaking at 37° C. and 200 rpm for 12 h to obtain cultured bacteria solution.

1.3.3 The cultured bacteria solution was transferred into 1.5 L LB fluid medium containing 50 μg/ml kanamycin by a volume ratio of 1:100, followed by culturing under shaking at 37° C. and 200 rpm to OD₆₀₀ₙₘ to 0.6. Then, Isopropyl-β-D-thiogalactopyranoside (IPTG) was added to a final concentration of 0.5 mM, and the bacteria were cultured under shaking at 16° C. and 200 rpm for 12 h, followed by centrifuging at 4° C. and 8000 rpm for 10 min to collect the bacterial pellet.

1.3.4 After step 1.3.3, the bacterial pellet was resuspended with the affinity solution A, then incubated on ice for 30 min, and the bacteria were ultrasonically broken in ice-water bath by a Φ6 probe of Ningbo Xinzhi ultrasonic breaker with 40% ultrasonic power, where the cycle program was set for breaking for 2 s, stopping for 3 s and the total program was for 30 min, followed by centrifuging at 4° C. and 15000 rpm for 30 min, and the supernatant was collected.

1.3.5 After step 4, the supernatant was purified rapidly with affinity chromatography, followed by dialyzing, where solutes and concentrations thereof in a dialysis buffer were 200 mM of KCl, 0.2 mM of EDTA, 5% Glyecrol and 20 mM of Tris-HCl; solvent of the buffer was water; pH value was 7.5; and the temperature was 25° C., and thus a crude recombinant phi29 DNA polymerase 1 was obtained.

Following the above steps, the material of recombinant plasmid pET28a-WT was individually replaced with the recombinant plasmid pET28a-T17P, recombinant plasmid pET28a-M97Y, recombinant plasmid pET28a-M97S, recombinant plasmid pET28a-M97R, recombinant plasmid pET28a-M97Q, recombinant plasmid pET28a-M97G, Plasmid pET28a-L123Y, recombinant plasmid pET28a-T140K, recombinant plasmid pET28a-T140H, recombinant plasmid pET28a-Y148P, recombinant plasmid pET28a-Y148E, recombinant plasmid pET28a-1158P, recombinant plasmid pET28a-T159A, recombinant plasmid pET28a-T203E, recombinant plasmid pET28a-Y224E, recombinant plasmid pET28a-F309S, recombinant plasmid pET28a-Y310N, recombinant plasmid pET28a-Y310G, recombinant plasmid pET28a-G320H, recombinant plasmid pET28a-G320E, recombinant plasmid pET28a-G320D, recombinant plasmid pET28a-G320C, recombinant plasmid pET28a-N344R, recombinant plasmid pET28a-V345E, recombinant plasmid pET28a-Y347G, recombinant plasmid pET28a-V509E, recombinant plasmid pET28a-E515W, recombinant plasmid pET28a-E515T, recombinant plasmid pET28a-E515S, recombinant plasmid pET28a-E515R, recombinant plasmid pET28a-E515N, recombinant plasmid Plasmid pET28a-E515I, recombinant plasmid pET28a-E515F, recombinant plasmid pET28a-R96E, recombinant plasmid pET28a-R96N, recombinant plasmid pET28a-R96S, recombinant plasmid pET28a-R96A, recombinant plasmid pET28a-R96G, recombinant plasmid pET28a-R96K, recombinant plasmid pET28a-M97C, recombinant plasmid pET28a-M97P, recombinant plasmid pET28a-M97V, recombinant plasmid pET28a-M97W, recombinant plasmid pET28a-M97N, recombinant plasmid pET28a-M97D, recombinant plasmid pET28a-M97E, recombinant plasmid pET28a-Q99V, recombinant plasmid pET28a-Q99E, recombinant plasmid pET28a-Q99T, recombinant plasmid pET28a-L123A, recombinant plasmid pET28a-L123C, recombinant plasmid pET28a-L123Q, recombinant plasmid pET28a-L123M, recombinant plasmid pET28a-L123N, recombinant plasmid pET28a-Q171E, recombinant plasmid pET28a-Q171K, recombinant plasmid Plasmid pET28a-T204K, recombinant plasmid pET28a-T213K, recombinant plasmid pET28a-G217K, recombinant plasmid pET28a-V250I, recombinant plasmid pET28a-V270R, recombinant plasmid pET28a-N344K, recombinant plasmid pET28a-Y369D, recombinant plasmid pET28a-Y369N, recombinant plasmid pET28a-K402L, recombinant plasmid pET28a-L416A, recombinant plasmid pET28a-E515H, recombinant plasmid pET28a-E515Q, recombinant plasmid pET28a-E515T and recombinant plasmid pET28a-1524V, where other steps were all unchanged, and thus crude recombinant phi29 DNA polymerases 2 to 71 were obtained sequentially.

### Example 2: Assay on Stabitity of the Crude Recombinant Phi29 DNA Polymerases Prepared in Example 1

The 71 crude recombinant phi29 DNA polymerases prepared in Example 1 and the dialysis buffer were assayed for the Tm value with a protein thermal shift assay kit (Life Technologies) individually. Specifically, a program was set and a reaction buffer was prepared according to the instructions of the protein thermal shift studies user guide. After the program was completed, the experimental results were input to protein thermal shift software for analysis, thereby obtaining the Tm values of each sample.

The recombinant phi29 DNA polymerase 1 was set as a positive control.

The dialysis buffer was set as a negative control.

Each sample was repeated in quadruplicate and averaged. Part of results is shown in Table 3. The results show that the crude recombinant phi29 DNA polymerases 2 to 34 each are of an increased Tm value to a certain degree as compared with the crude recombinant phi29 DNA polymerase 1, indicating that the crude recombinant phi29 DNA polymerases 2 to 34 exhibit an improved stability to a certain degree.

TABLE 3

| Crude recombinant phi29 | | Crude recombinant phi29 | |
|---|---|---|---|
| DNA polymerase | Tm (° C.) | DNA polymerase | Tm (° C.) |
| Crude recombinant phi29 | 49.60677 | Crude recombinant phi29 | 48.22635 |
| DNA polymerase 26 | | DNA polymerase 6 | |
| Crude recombinant phi29 | 48.38237 | Crude recombinant phi29 | 48.18446 |
| DNA polymerase 18 | | DNA polymerase 1 | |
| Crude recombinant phi29 | 48.24233 | Crude recombinant phi29 | 48.2925 |
| DNA polymerase 19 | | DNA polymerase 7 | |
| Crude recombinant phi29 | 48.91264 | Crude recombinant phi29 | 49.04076 |
| DNA polymerase 16 | | DNA polymerase 8 | |
| Crude recombinant phi29 | 48.54876 | Crude recombinant phi29 | 49.07857 |
| DNA polymerase 11 | | DNA polymerase 34 | |
| Crude recombinant phi29 | 48.66696 | Crude recombinant phi29 | 48.20793 |
| DNA polymerase 12 | | DNA polymerase 13 | |
| Crude recombinant phi29 | 48.87052 | Crude recombinant phi29 | 48.22497 |
| DNA polymerase 27 | | DNA polymerase 20 | |
| Crude recombinant phi29 | 48.88358 | Crude recombinant phi29 | 48.21473 |
| DNA polymerase 25 | | DNA polymerase 21 | |
| Crude recombinant phi29 | 48.49379 | Crude recombinant phi29 | 48.61902 |
| DNA polymerase 15 | | DNA polymerase 22 | |
| Crude recombinant phi29 | 48.61609 | Crude recombinant phi29 | 48.46351 |
| DNA polymerase 2 | | DNA polymerase 23 | |
| Crude recombinant phi29 | 49.30812 | Crude recombinant phi29 | 49.03835 |
| DNA polymerase 14 | | DNA polymerase 17 | |
| Crude recombinant phi29 | 48.55235 | Crude recombinant phi29 | 48.96379 |
| DNA polymerase 9 | | DNA polymerase 28 | |
| Crude recombinant phi29 | 48.91756 | Crude recombinant phi29 | 48.24366 |
| DNA polymerase 10 | | DNA polymerase 29 | |
| Crude recombinant phi29 | 48.27392 | Crude recombinant phi29 | 48.68517 |
| DNA polymerase 24 | | DNA polymerase 30 | |
| Crude recombinant phi29 | 49.18874 | Crude recombinant phi29 | 48.68651 |
| DNA polymerase 3 | | DNA polymerase 31 | |
| Crude recombinant phi29 | 49.12807 | Crude recombinant phi29 | 48.88325 |
| DNA polymerase 5 | | DNA polymerase 32 | |
| Crude recombinant phi29 | 49.45142 | Crude recombinant phi29 | 48.23149 |
| DNA polymerase 4 | | DNA polymerase 33 | |

### Example 3: Assary on Specific Enzyme Activity of the Crude Recombinant Phi29 DNA Polymerases Prepared in Example 1

Each of the 71 crude recombinant phi29 DNA polymerases prepared in Example 1 was subjected to the assay on specific enzyme activity.

3.1 The crude recombinant phi29 DNA polymerases were assayed for protein concentration with a BCA kit, followed by diluting with the dialysis buffer, thereby obtaining respective diluents of the recombinant phi29 DNA polymerases with a concertration of 5 μg/ml.

Solutes and concentrations thereof in the dialysis buffer were 20 mM of Tris-HCl, 200 mM of KCl, 2 mM of DTT, 0.2 mM of EDTA and 5% Glyecrol; and solvent was water; and pH value was 7.4.

3.2 A reaction mixture was prepared. The reaction mixture was in a volume of 80.8 μl, including DTT, (NH₄)₂SO₄, MgCl₂, dNTP Mixture, RCA Primer (i.e., Ad153 make DNB primer, the product of Invitrogen, Cat. No. R082), 6 ng of single-stranded circular DNA template 153Ad ssDNA and 50 mM of Tris-HCl buffer with pH7.5. In the reaction mixture, the concentration of DTT was 4 mM, the concentration of (NH₄)₂SO₄ was 10 mM, the concentration of MgCl₂ was 10 mM, the concentration of dNTP Mixture was 50 nM and the concentration of RCA Primer was 2 pM.

3.3 The reaction mixture was placed in a PCR amplifier for primer-template hybridization, and the program was set as follows: 95° C. for 1 min, 65° C. for 1 min, 40° C. for 1 min, and the temperature of the hot cover was set at 102° C. When the temperature reached 4° C., the PCR tube was taken out and placed on ice, added with 1 μl of the diluent of the recombinant phi29 DNA polymerase, followed by shaking and mixing with a vortex shaker. With a temporary centrifugation, the PCR tube was placed in the PCR amplifier for reaction, and the reaction conditions were 30° C. for 60 min and the temperature of the hot cover was set at 65° C. After the reaction was completed, 5 μl of EDTA solution at a concentration of 0.5 M was added to stop the reaction. After shaking and mixing, a reaction product was obtaining.

3.4 The reaction product obtained in step 3.3 was assayed for concentration with the Qubit fluorometer 3.0, according to the instructions of the Qubit ssDNA assay kit. IU enzyme activity is defined as the amount of enzyme required for producing DNB based on 10 nmol dNTP at 30° C. for 60 min. The the crude recombinant phi29 DNA polymerases were further assayed for specific enzyme activity.

Part of the results is shown in Table 4. The results show that the crude recombinant phi29 DNA polymerase 3, the crude recombinant phi29 DNA polymerase 4, the crude recombinant phi29 DNA polymerase 5 and the crude recombinant phi29 DNA polymerases 34 to 71 each are of an increased specific enzyme activitiy to a certain degree as compared with the crude recombinant phi29 DNA polymerase 1, indicating that the crude recombinant phi29 DNA polymerase 3, the crude recombinant phi29 DNA polymerase 4, the crude recombinant phi29 DNA polymerase 5 and the crude recombinant phi29 DNA polymerases 34 to 71 each exhibit an improved DNA polymerase activity to a certain degree.

TABLE 4

| | Specific | | Specific |
|---|---|---|---|
| | enzyme | | enzyme |
| Crude recombinant phi29 | activity | Crude recombinant phi29 | activity |
| DNA polymerase | (U/μg) | DNA polymerase | (U/μg) |
| Crude recombinant phi29 | 27.86966 | Crude recombinant phi29 | 25.01471 |
| DNA polymerase 5 | | DNA polymerase 48 | |
| Crude recombinant phi29 | 37.38322 | Crude recombinant phi29 | 37.27798 |
| DNA polymerase 3 | | DNA polymerase 41 | |
| Crude recombinant phi29 | 27.97547 | Crude recombinant phi29 | 39.54949 |
| DNA polymerase 61 | | DNA polymerase 42 | |
| Crude recombinant phi29 | 32.28242 | Crude recombinant phi29 | 29.82229 |
| DNA polymerase 66 | | DNA polymerase 43 | |
| Crude recombinant phi29 | 18.99764 | Crude recombinant phi29 | 25.93127 |
| DNA polymerase 35 | | DNA polymerase 44 | |
| Crude recombinant phi29 | 29.17352 | Crude recombinant phi29 | 30.63974 |
| DNA polymerase 56 | | DNA polymerase 45 | |
| Crude recombinant phi29 | 27.19059 | Crude recombinant phi29 | 56.83838 |
| DNA polymerase 58 | | DNA polymerase 46 | |
| Crude recombinant phi29 | 27.57224 | Crude recombinant phi29 | 42.18217 |
| DNA polymerase 59 | | DNA polymerase 47 | |
| Crude recombinant phi29 | 26.18703 | Crude recombinant phi29 | 31.52057 |
| DNA polymerase 64 | | DNA polymerase 51 | |
| Crude recombinant phi29 | 28.0683 | Crude recombinant phi29 | 35.26901 |
| DNA polymerase 63 | | DNA polymerase 52 | |
| Crude recombinant phi29 | 21.52907 | Crude recombinant phi29 | 64.68987 |
| DNA polymerase 36 | | DNA polymerase 53 | |
| Crude recombinant phi29 | 22.02841 | Crude recombinant phi29 | 23.80637 |
| DNA polymerase 57 | | DNA polymerase 54 | |
| Crude recombinant phi29 | 24.79935 | Crude recombinant phi29 | 34.63575 |
| DNA polymerase 60 | | DNA polymerase 55 | |
| Crude recombinant phi29 | 28.91615 | Crude recombinant phi29 | 25.04765 |
| DNA polymerase 65 | | DNA polymerase 50 | |
| Crude recombinant phi29 | 33.68722 | Crude recombinant phi29 | 20.20621 |
| DNA polymerase 67 | | DNA polymerase 68 | |
| Crude recombinant phi29 | 22.68731 | Crude recombinant phi29 | 16.16717 |
| DNA polymerase 71 | | DNA polymerase 38 | |
| Crude recombinant phi29 | 24.50766 | Crude recombinant phi29 | 18.00866 |
| DNA polymerase 34 | | DNA polymerase 39 | |
| Crude recombinant phi29 | 19.88544 | Crude recombinant phi29 | 19.72106 |
| DNA polymerase 4 | | DNA polymerase 40 | |
| Crude recombinant phi29 | 19.72727 | Crude recombinant phi29 | 16.17894 |
| DNA polymerase 62 | | DNA polymerase 49 | |
| Crude recombinant phi29 | 13.88 | Crude recombinant phi29 | 17.54294 |
| DNA polymerase 1 | | DNA polymerase 69 | |
| Crude recombinant phi29 | 39.47822 | Crude recombinant phi29 | 69.588 |
| DNA polymerase 37 | | DNA polymerase 70 | |

### Example 4: Preparation of a Phi29 DNA Polymerase with Combined Mutations and Assay on the Stability and Specific Enzyme Activity

### 4.1 Construction of a Recombinant Phi29 DNA Polymerase (i.e., the Phi29 DNA Polymerase with Combined Mutations)

The phi29 DNA polymerase with combined mutations were constructed by a DNA shuffling method or multi-site directed mutagenesis, based on the mutant sites provided in Example 1 and the known mutant sites disclosed in the literature.

The specific steps of the DNA shuffling method were as follows.

4.1.1.1 The template for shuffling was amplified with PCR (forward primer: 5′-CTGGTGCCGCGCGGCAGCCATATG-3′; reverse primer: 5′-CTCGAATTCGGATCCTCACTTGA-3′). The PCR product was then recovered via cutting the gel.

4.1.1.2 A digestion reaction with DNase I enzyme was performed according to the steps shown in Table 5.

TABLE 5

| | Reagent | Volume (μl) | Conditions |
|---|---|---|---|
| Step 1 | 10 × DNaseI buffer | 5.0 | 15 min 15° C. |
| | DNA1 | 22.5 | (equilibrium) |
| | (concertration at | (concertration at | |
| | about 40 ng/μl) | about 900 ng/μl) | |
| | DNA2 | 22.5 | |
| | (concertration at | (concertration at | |
| | about 40 ng/μl) | about 900 ng/μl) | |
| Step 2 | (0.4 U/μL) DNaseI | 0.75 (0.3 U) | 1.5 min 15° C. |
| | | | (reacting) |
| Step 3 | 0.5M EDTA | 0.5 | 90° C. 10 min |
| | | | (stopping and |
| | | | melting) |

4.1.1.3 After the step 4.1.1.2, the digested DNA fragments were recovered with M280 magnetic beads, then washed with 75% (v/v) ethanol aqueous solution twice, and dissolved with ddH₂O.

4.1.1.4 After the step 4.1.1.3, the fragmented DNA fragments were subjected to shuffling recombination with PCR.

The reaction system is shown in Table 6.

TABLE 6

| Reagent | Volume (μl) |
|---|---|
| ddH₂O | (21.5-DNA) |
| DNA | 0.25/0.5/1.0 |
| 10 × pfu buffer | 2.5 |
| 10 mM dNTP | 0.5 |
| Pfu polymerase | 0.5 |

The reaction program was set as: (i) 95° C. for 3 min; (ii) 95° C. for 30 s, 65° C. for 30 s, 72° C. for 1 min, and (ii) was proformed for 45 cycles; and (iii) 72° C. for 3 min; and (iv) 4° C. for hold.

4.1.1.5 After the step 4.1.1.4, enrichment by a second amplification was performed by taking the recombined fragments as the template.

The reaction system is shown in Table 7.

TABLE 7

| Reagent | Volume (μl) |
|---|---|
| DNA | 2.5 |
| ddH₂O | 18.0 |
| 10 × pfu buffer | 2.5 |
| 10 mM dNTP | 0.5 |
| 10 μM Primer 1 | 0.5 |
| (mix of the forward primers in Table 2) | |
| 10 μM Primer 2 | 0.5 |
| (mix of the reverse primers in Table 2) | |
| 2.5 U/μLPfu polymerase | 0.5 |

The reaction program was set as: (i) 95° C. for 3 min; (ii) 94° C. for 30 s, 60° C. for 30 s, 72° C. for 1 min and 40 s, and (ii) was proformed for 60 cycles; and (iii) 72° C. for 7 min.

With the construction of a mutant with 5 mutation sites as an example, the specific steps of the multi-site directed mutagenesis were as follows.

4.1.2.1 Primer design: the mutation site was designed in the middle of the primer flank by about 15 nt, and a pair of reversely complementary primers was desiged for each mutation site.

4.1.2.2 Prepartion of the reaction system: the reaction system was in a volume of 25 μl, including 12.5 μL of 2×KAPA HiFi HS Ready Mix, 3.5 μL of FW primer at a concertration of 2 μM which was a mix of 5 forward primers in total where each forward primer was accounted for 0.7 μl; 3.5 μL RE primer at a concertration of 2 μM which was a mix of 5 reverse primers in total where each reverse primer was accounted for 0.7 μl; 75 ng of the template and H₂O.

4.1.2.3 PCR amplification was proformed with the PCR reaction system above.

The reaction program was set as: (i) 95° C. for 3 min; (ii) 98° C. for 20 s, 65° C. for 15 s, 72° C. for 7 min, and (ii) was proformed for 19 cycles; (iii) 72° C. for 10 min; and (iv) 12° C. for hold.

4.1.2.4 After the step 4.1.2.3, the PCR product was added with 1 μL of dpn1 enzyme for digestion for 2 h at 37° C., then transformed into E. coli DH5a competent cells, followed by spreading on medium plates and culturing at 37° C. overnight. Single clones were picked and plasmids therein were extracted for sequencing.

### 4.2 High-Throughput Screening of the Phi29 DNA Polymerase with Combined Mutations Constructed in Example 1

The phi29 DNA polymerase with combined mutations constructed in Example 1 was subjected to high-throughput screening by the isothermal compartmentalization self-replication (iCSR). Similar to the compartmentalization self-replication (CSR) where a plasmid for the phi29 DNA polymerase is self-replicated by means of its own activity of strand displacement, the recombinant phi29 DNA polymerase with higher activity was enriched through several rounds of screenings based on different recombinant phi29 DNA polymerases with different activities producing different amounts of amplified DNA. Specific steps performed were as follows.

### 4.2.1 Primer Design

3 pairs of primers were required during the whole process, where a primer pair for iCSR was used for the amplification in the iCSR process, and the 3′ end of the primer pair for iCSR was thio-modified to prevent digestion by the exonuclease in the cell; and primer pairs for Insertion and vector amplification were used for amplification of an insert and a template in in-fusion reactions, respectively.

The primer pair for iCSR included Primer 1 (5′-TTGAGGCCGTTGAGCACC-3′) with thio-modification at the 3′ end, and Primer 2 (5′-CCGGATATAGTTCCTCCTTTCAG-3′) with thio-modification at the 3′ end.

The primer pair for Insertion included Primer 3 (S′-AATGTATAGCTGCGACTTTGAAACCA-3′) and Primer 4 (5′-TAGAGGCCCCAAGGGGTTAT-3′).

The primer pair for vector amplification included Primer 5 (5′-ATAACCCCTTGGGGCCTCTA-3′) and Primer 6 (5′-TGGTTTCAAAGTCGCAGCTATACAT-3′).

### 4.2.2 Cell Transformation and Protein Expression

The constracted library of the recombinant phi29 DNA polymerases was transformed into E. coli BL21 competent cells, and the cells incubated at 37° C. were transferred into 2 ml LB fluid medium containing kanamycin directly without spreading on the medium plate, followed by culturing at 37° C. overnight. On the second day, the LB fluid medium containing the cells were transferred into fresh LB liquid medium containing kanamycin at a ratio of 1:200 and cultured at 37° C. for 3 h, and then IPTG was added to a final concentration of 0.5 mM to induce the cells at 16° C. overnight.

### 4.2.3 Preparation of a Reaction System of iCSR

4.2.3.1 Preparation of a reaction buffer: the reaction buffer was in a volume of 2 ml, including 200 μL of 10×phi29 reaction buffer, 40 μL of 500 μM Exo-resistant primer mix, 60 μL of 10 μM primer 1, 60 μL of 10 μM primer 2, 40 μL of 25 mM dNTPmix and 1600 μL of NFH₂O.

### 4.2.3.2 Preparation of Cells

a. 0.45 mL of 1×phi29 reaction buffer was mixed with 0.05 mL of lysozyme (10 mg/mL), followed by preheating in metal bath at 30° C., thereby obtaining a cell lysis buffer.

b. Assay of an OD value and Calculation of a final dilution volume

The the E. coli cells were assayed for the OD value. The number of cells=OD×8×10⁸×2=16×OD×10⁸, estimated based on that the concentration was 8×10⁸ cells/mL when OD=1, the dilution volume was set as VmL.

Assuming that the diameter of a microdroplet generated was about 21 μm, the volume of a single microdroplet was 5 μL, and the volume of the bacterial channel was 2.5 μL according to the same flow rate in the bacterial channel and the buffer channel.

$$λ = 16 \times OD \times {10}^{8} cells / VmL \times 2.5 pL = 16 \times 2.5 \times OD \times {10}^{8} \times {10}^{- 9} / V = 4 \times OD / V$$

If λ=0.2, there was 1% of the microdroplets containing two single cells while 16% of the microdroplets containing one single cell, and V=20×OD.

c. Cell preparation

The induced E. coli cells were centrifuged at 12,000 rpm for 1 min with supernatant discard. The pellet was resuspended with 1 mL of 1×phi29 reaction buffer and washed twice, then centrifuged at 12,000 rpm for 1 min, resuspended with 0.5 mL of cell lysis buffer, and incubated at 30° C. for 5 min with shaking at 300 rpm. The cells were then recovered by centrifugation at 12,000 rpm for 1 min, and then resuspended and diluted with V mL of the 1×phi29 reaction buffer, followed by placing on ice.

### 4.2.4 Preparation of Microdroplet

The diameter of the microdroplet was controlled at about 20 μm. The generated microedroplets were collected on ice, and about 500 μL of microdroplets were collected.

### 4.2.5 iCSR Reaction

The collected microdroplets were aliquoted into PCR tubes at 30 μL/tube on ice. The recombinant phi29 DNA polymerases capable of reacting at a high temperature were tested and screened at the gradient temperatures in the PCR amplifier in this experiment, where the experimental conditions were set as 37° C.-55° C. for 2 h/16 h, and then 85° C. for 15 min for thermal inactivating for the recombinant phi29 DNA polymerases.

### 4.2.6 Emulsion Breaking

4.2.6.1 The same number of PhaseLock tubes as the PCR tubes were centrifuged at 16,000 g for 30 s for a pretreatment.

4.2.6.2 Isopyknic PFO demulsifier was added to the the PCR tubes after the reaction of step 4.2.5, followed by transferring to a 1.5 mL EP tube after fully mixed and centrifuging at 14,000 rpm for 10 min. Then all the liquid was transferred into the phaseLock tubes and centrifuged at 16,000 g for 5 min. The upper liquid was transferred into new 8-strip PCR tubes.

### 4.2.7 Enzyme Digestion and Quantification with Qubit

9 μL of iCSR product was placed in the new 8-strip PCR tube and directly added with 0.5 μL of dpnI enzyme to disgest the plasmid as the template and 0.5 μL of Xbal enzyme to cut the amplified product into single copies, followed by digesting at 37° C. for 2 h. Then, 1 μL of the disgested product was taken to quantification with the Qubit dsDNA HS assay kit.

### 4.2.8 Second Amplification

A KAPA HiFi HotStart PCR Kit was used for amplification. The amount of reaction buffer should be adjusted as Mg²⁺ had been accumulated in the previous steps in view of no purification steps invloved, and a ready mix should not be used.

The reaction system was in a volume of 50 μL, including 8 μL of 5×HiFidelity buffer, 1.5 μL of 10 μM FW primer, 1.5 μL of 10 μM RE primer, 2 μL of template DNA taken from the tubes in step 3.7, 1.5 μL of 10 mM dNTP mix, 34.5 μL of NFH₂O and 1 μL of HiFi Enzyme.

Additionally, the vector taken as the template was amplified with ReadyMix as normal.

The reaction conditions were set as: (i) 95° C. for 3 min; (ii) 98° C. for 20 s, 65° C. for 15 s, 72° C. for 2 min, and (ii) was proformed for 35 cycles; and (iii) 72° C. for 10 min; and (iv) 4° C. for hold.

### 4.2.9 Recovery Via Gel

The PCR product added with 6×loading dye was subjected to agarose gel electrophoresis and was recovered after cutting the gel, according to the steps of the gel extraction kit. The recovered product was quantified.

### 4.2.10 In-Fusion Reaction

According to the instructions of the In-Fusion HD Cloning Kit, a recommended input amount is 50-100 ng when the insert length is 0.5-10 kb, and is 50-100 ng when the length of the vector is less than 10 kb. When there is only one insert, the recommended molar ratio of the insert and vector is 2:1. Accordingly, the reaction system in this Example was 10 μL, including 50 ng of purified PCR fragment, 78 ng of linearized vector, 2 μL of 5×In-fusion HD Enzyme mix and NFH₂O.

The reaction conditions were set as: (i) 50° C. for 15 min; and (ii) 4° C. for hold.

### 4.2.11 Transformation and Sequencing

The in-fusion product was directly transformed into KRX/BL21 competent cells (if a high transformation rate is required, the in-fusion product could be also transformed into DH5a cells first, and then transformed into BL21 cells after plasmid extraction), followed by transferring into LB liquid medium containing kanamycin directly. On the second day, the bacteria solution was prepared for expression inducing for the next round of screening. After the second round of screening, the bacteria on the plates were sequenced.

After the above steps, four recombinant phi29 DNA polymerases with combined mutations having higher activity were obtained, namely a recombinant phi29 DNA polymerase with T213K/L416A/V509E mutations, a recombinant phi29 DNA polymerase with M97T/Y224K/E515S mutations, a recombinant phi29 DNA polymerase with L123Q/T159A/Y347G mutations and a recombinant phi29 DNA polymerase with R96S/L123P/Y224K/L416A/E515S mutations.

The recombinant phi29 DNA polymerase with T213K/L416A/V509E mutations differs with the phi29 DNA polymerase shown in SEQ ID NO: 2 only in that T at position 213 of the latter was substituted by K, L at position 416 was substituted by A, and V at position 509 was substituted by E.

The recombinant phi29 DNA polymerase with M97T/Y224K/E515S mutations differs with the phi29 DNA polymerase shown in SEQ ID NO: 2 only in that M at position 97 of the latter was substituted by T, Y at position 224 was substituted by K, and E at position 515 was substituted by S.

The recombinant phi29 DNA polymerase with L123Q/T159A/Y347G mutations differs with the phi29 DNA polymerase shown in SEQ ID NO: 2 only in that L at position 123 of the latter was substituted by Q, T at position 159 was substituted by A, and Y at position 347 was substituted by G.

The recombinant phi29 DNA polymerase with R96S/L123P/Y224K/L416A/E515S mutations differs with the phi29 DNA polymerase shown in SEQ ID NO: 2 only in that R at position 96 of the latter was substituted by S, L at position 123 was substituted by P, Y at position 224 was substituted by K, L at position 416 was substituted by A, and E at position 515 was substituted by S.

### 4.3 Obtaintion of the Recombinant Phi29 DNA Polymerase with Combined Mutations

The phi29 DNA polymerase shown in SEQ ID NO: 2, the recombinant phi29 DNA polymerase with T213K/L416A/V509E mutations, the recombinant phi29 DNA polymerase with M97T/Y224K/E515S mutations, the recombinant phi29 DNA polymerase with L123Q/T159A/Y347G mutations and the recombinant phi29 DNA polymerase with R96S/L123P/Y224K/L416A/E515S mutations were prepared.

### 4.4 Assay on Stabitity of the Phi29 DNA Polymerase with Combined Mutations

According to the method in Example 2, the recombinant phi29 DNA polymerase with T213K/L416A/V509E mutations, the recombinant phi29 DNA polymerase with M97T/Y224K/E515S mutations, the recombinant phi29 DNA polymerase with L123Q/T159A/Y347G mutations and the recombinant phi29 DNA polymerase with R96S/L123P/Y224K/L416A/E51SS mutations were detected for the Tm values.

The detection results are shown in Table 8. The results show that three recombinant phi29 DNA polymerases with respective combined mutations were of a significantly increased Tm value as compared with the phi29 DNA polymerase shown in SEQ ID NO: 2, that is, the three recombinant phi29 DNA polymerases with respective combined mutations each exhibit a significantly improved stability.

TABLE 8

| | Tm(° C.) |
|---|---|
| phi29 DNA polymerase shown in SEQ ID NO: 2 | 48.5 |
| recombinant phi29 DNA polymerase with | 49.4 |
| T213K/L416A/V509E mutations | |
| recombinant phi29 DNA polymerase with | 50.8 |
| M97T/Y224K/E515S mutations | |
| recombinant phi29 DNA polymerase with | 46.1 |
| L123Q/T159A/Y347G mutaions | |
| recombinant phi29 DNA polymerase with | 51.6 |
| R96S/L123P/Y224K/L416A/E515S mutations | |

### 4.5 Assay on Specific Enzyme Activity of the Recombinant Phi29 DNA Polymerase with Combined Mutations

According to the method in Example 3, the recombinant phi29 DNA polymerase with T213K/L416A/V509E mutations, the recombinant phi29 DNA polymerase with M97T/Y224K/E515S mutations, the recombinant phi29 DNA polymerase with L123Q/T159A/Y347G mutations and the recombinant phi29 DNA polymerase with R96S/L123P/Y224K/L416A/E515S mutations were detected the specific enzyme activity.

The detection results are shown in Table 9. The results show that two recombinant phi29 DNA polymerases with respective combined mutations each were of a significantly increased specific enzyme activity as compared with the phi29 DNA polymerase shown in SEQ ID NO: 2, that is, the two recombinant phi29 DNA polymerases with respective combined mutations each exhibit a significantly improved specific enzyme activity.

TABLE 9

| | Specific enzyme |
|---|---|
| | activity (U/μg) |
| phi29 DNA polymerase shown in SEQ ID NO: 2 | 43 |
| recombinant phi29 DNA polymerase with | 52 |
| T213K/L416A/V509E mutations | |
| recombinant phi29 DNA polymerase with | 55 |
| M97T/Y224K/E515S mutations | |
| recombinant phi29 DNA polymerase with | 9 |
| L123Q/T159A/Y347G mutations | |
| recombinant phi29 DNA polymerase with | 28 |
| R96S/L123P/Y224K/L416A/E515S mutations | |

It can be seen that the recombinant phi29 DNA polymerase with T213K/L416A/V509E mutations and the recombinant phi29 DNA polymerase with M97T/Y224K/E515S mutations each have higher stability and specific enzyme activity, showing better effects. The recombinant phi29 DNA polymerase with R96S/L123P/Y224K/L416A/E515S mutations has high stability but is slightly poor in specific enzyme activity. The recombinant phi29 DNA polymerase L123Q/T159A/Y347G mutations is poor in both stability and specific enzyme activity.

### INDUSTRIAL APPLICATION

Compared with the existing phi29 DNA polymerase, 73 recombinant phi29 DNA polymerases with significantly improved stability and/or specific enzyme activity were prepared in embodiments of the present disclosure. These recombinant phi29 DNA polymerases not only have improved thermal stability, the polymerization activity and processivity are also improved. When the recombinant phi29 DNA polymerases prepared in embodiments of the present disclosure are used in amplification or sequencing, DNA can be efficiently and continuously synthesized, and the reaction efficiency is high. The present disclosure has important application value.

1. A protein of C1) or C2), wherein
C1) is a protein having DNA polymerase activity obtained by substituting an amino acid residue of a phi29 DNA polymerase at the following positions:
at least one of position 97, position 515 and position 224; or
at least one of position 213, position 416 and position 509;
C2) is a fusion protein obtained by attaching a tag to the N-terminus or/and C-terminus of the protein of C1),
wherein an amino acid sequence of the phi29 DNA polymerase is set forth in SEQ ID NO: 2.

2. The protein according to claim 1, wherein
M at position 97 is substituted with Y, S, R, Q, G, C, P, V, W, N, D, E or T;
T at position 213 is substituted with K;
Y at position 224 is substituted with E or K;
L at position 416 is substituted with A;
V at position 509 is substituted with E;
E at position 515 is substituted with W, T, S, R, N, I, F, H, Q or T.

3. The protein according to claim 1, wherein the protein is of at least one of higher stability and specific enzyme activity than that of the phi29 DNA polymerase.

4. The protein according to claim 1, wherein the protein is a recombinant phi29 DNA polymerase with T213K/L416A/V509E mutations or a recombinant phi29 DNA polymerase with M97T/Y224K/E515S mutations, wherein
the recombinant phi29 DNA polymerase with T213K/L416A/V509E mutations is a protein obtained by substituting T at position 213 with K, L at position 416 with A and V at position 509 with E, from the N-terminus of SEQ ID NO: 2;
the recombinant phi29 DNA polymerase with M97T/Y224K/E515S mutations is a protein obtained by substituting M at position 97 with T, Y at position 224 with K and E at position 515 with S, from the N-terminus of SEQ ID NO: 2.

5. The protein according to claim 1, wherein the protein is any one of the following proteins:
protein a2 is a protein obtained by substituting M at position 97 with Y from the N-terminus of SEQ ID NO: 2;
protein a26 is a protein obtained by substituting V at position 509 with E from the N-terminus of SEQ ID NO: 2;
protein a33 is a protein obtained by substituting E at position 515 with F from the N-terminus of SEQ ID NO: 2;
protein a58 is a protein obtained by substituting T at position 213 with K from the N-terminus of SEQ ID NO: 2.

6. The protein according to claim 1, wherein the protein is any one of the following proteins:
protein b2 is a protein obtained by substituting M at position 117 with Y from the N-terminus of SEQ ID NO: 4;
protein b26 is a protein obtained by substituting V at position 529 with E from the N-terminus of SEQ ID NO: 4;
protein b33 is a protein obtained by substituting E at position 535 with F from the N-terminus of SEQ ID NO: 4;
protein b58 is a protein obtained by substituting T at position 233 with K from the N-terminus of SEQ ID NO: 4.

7. A nucleic acid molecule encoding a protein of C1) or C2), wherein
C1) is a protein having DNA polymerase activity obtained by substituting an amino acid residue of a phi29 DNA polymerase at the following positions:
at least one of position 97, position 515 and position 224; or
at least one of position 213, position 416 and position 509;
C2) is a fusion protein obtained by attaching a tag to the N-terminus or/and C-terminus of the protein of C1),
wherein an amino acid sequence of the phi29 DNA polymerase is set forth in SEQ ID NO: 2.

8.-15. (canceled)

16. The protein according to claim 1, wherein
M at position 97 is substituted with T or Y, E at position 515 is substituted with F or S, and Y at position 224 is substituted with K; or
T at position 213 is substituted with K, L at position 416 is substituted with A, and V at position 509 is substituted with E.

17. The protein according to claim 1, wherein the protein of C1) is substituted at at least one of position 97 and position 515.

18. The protein according to claim 1, wherein the protein of C1) is substituted at at least one of position 97 and position 224.

19. The protein according to claim 1, wherein the protein of C1) is substituted at at least one of position 224 and position 515.

20. The protein according to claim 1, wherein the protein of C1) is substituted at at least one of position 97, position 224 and position 515.

21. The protein according to claim 1, wherein the protein of C1) is substituted at at least one of position 213 and position 416.

22. The protein according to claim 1, wherein the protein of C1) is substituted at at least one of position 213 and position 509.

23. The protein according to claim 1, wherein the protein of C1) is substituted at at least one of position 416 and position 509.

24. The protein according to claim 1, wherein the protein of C1) is substituted at at least one of position 213, position 416 and position 509.

25. An amplification method by use of the protein of C1) or C2), wherein
C1) is a protein having DNA polymerase activity obtained by substituting an amino acid residue of a phi29 DNA polymerase at the following positions:
at least one of position 97, position 515 and position 224; or
at least one of position 213, position 416 and position 509;
C2) is a fusion protein obtained by attaching a tag to the N-terminus or/and C-terminus of the protein of C1),
wherein an amino acid sequence of the phi29 DNA polymerase is set forth in SEQ ID NO: 2.

26. The amplification method according to claim 25, wherein the amplification is second strand amplification, single cell amplification or plasmid amplification.
